(19) United States
(12) Patent Application Publication
(10) Pub. No.: US 2006/0134011 A1
(43) Pub. Date: Jun. 22, 2006

(54) Oral and dental composition

(76) Inventor: Patrick John Shanahan, Claremont (AU)

Correspondence Address: NIXON & VANDERHYE, PC, 901 NORTH GLEBE ROAD, 11TH FLOOR, ARLINGTON, VA, 22203, US

(21) Appl. No.: 10/515,821

(22) PCT Filed: Jul. 22, 2003
(86) PCT No.: PCT/AU03/00928

§ 371 Date: Aug. 1, 2005

(30) Foreign Application Priority Data
Jul. 29, 2002 (AU) 2002950426

(51) Int. Cl. A61K 8/73

(52) U.S. Cl. 424/49

(57) ABSTRACT

The invention provides an oral and dental composition comprising an effective antibacterial amount of a combination of a physiologically acceptable divalent metal ion; a quaternary ammonium compound; chitosan or a chitosan derivative in a form to facilitate interaction with the other two components of the combination; and a physiologically acceptable excipient.

### FIELD OF THE INVENTION

The invention relates to an oral and dental composition, in particular for the prevention and alleviation of a number of oral and dental conditions, and to a method for use therewith.

### BACKGROUND ART

Oral and dental disease is a very common problem occurring in the community.

The major dental diseases are dental caries or tooth decay, and periodontal (gum) disease. Dental plaque is considered to be the initiator of dental disease.

The exact cause of dental disease is unknown but it is thought to be a bacterial process. In the case of periodontal disease, the precursor is gingivitis (inflammation of the gums) caused by the harmful breakdown products produced by bacteria in dental plaque. Thus, bacteria in the mouth play a major role in dental and oral disease.

There are many other common oral and dental conditions and these include mouth ulcers, bad breath, sensitive teeth and xerostomia (dry mouth) caused by disease or medical treatment and medications. In some of these conditions, the mouth and gums become inflamed, painful and very sensitive.

The accepted method of preventing dental disease is based on the mechanical removal of plaque using a toothbrush and toothpaste. Most toothpastes are abrasive and have limited antibacterial effect.

Often a toothbrush cannot be used because of severe medical conditions, such as behavioural problems associated with dementia, swallowing problems following a stroke, road trauma, and terminal conditions among others.

Most current oral hygiene products are focussed on tooth appearance and fresh breath. Other oral hygiene products are designed to address a specific need.

Many of the anti-bacterial agents used in oral and dental hygiene products, such as chlorhexidine, have a number of unpleasant side effects.

People are living longer and retaining their teeth, with a resulting increase in the prevalence of dental and oral diseases. Dementia in the elderly is increasing. Carers and nurses, especially those caring for dementia patients, have difficulty in managing the daily oral and dental health care needs of patients.

Thus, there is a need for a multi-purpose oral and dental hygiene product for use in both the prevention and alleviation of a range of oral and dental conditions. There is a need for a product that can be easily administered and that has effective anti-bacterial activity without any unpleasant side effects.

It is against the above background that the present invention has been developed.

It should be understood that throughout this specification the phrase “an oral and dental composition” is understood to mean a composition for use in either one of an oral or a dental application, or for use in both oral and dental applications. It is to be understood that the composition is intended for use in a human or animal subject.

### DISCLOSURE OF THE INVENTION

In work leading up to the invention, the inventor believed that the addition of chitosan when formulated in a dentifrice composition, could provide beneficial properties in the prevention and treatment of dental disease.

Chitosan is a polysaccharide, derived from the deacetylation of chitin. Chitin is a naturally occurring polymer and nature's second most abundant polymer after cellulose.

The inventor's initial formulations comprised chitosan in the form of a powder. However, these initial formulations comprising powdered chitosan did not provide the antibacterial activity desired. It was believed that this poor performance was due to the insolubility of the powdered chitosan making it unavailable to interact or react with the other components in the formulations tested.

However, in further developmental work, the inventor found unexpected and surprising beneficial properties, if chitosan was present in a composition in a form that increased its bioavailability and ability to interact or react with other components in a suitable composition. The resulting compositions produced greatly enhanced performance in the treatment of a range of oral and dental conditions.

It is believed that the presence of chitosan in a chemically available and bioavailable form assists the other components to maximise their antibacterial effects. It is thought that this occurs by the chitosan forming a layer on a bacterial cell wall which assists in allowing penetration of the other components having antibacterial effect thereby substantially increasing their antibacterial effect due to the ease of exposure to the bacterial cell wall. In this way, the compositions of the invention have unexpected enhanced antibacterial effects for a range of oral and dental conditions.

It is also believed that chitosan in the composition of the invention assists in forming a protective coating on tooth structures and soft tissues thereby delaying or preventing the onset of bacterial infections in the gums and mouth, and assisting in reducing plaque build-up. It is further believed that chitosan in the form as used in the invention is able to interact with the physiology of the saliva in the mouth of a subject thereby assisting in producing the unexpected and beneficial antibacterial effects.

Thus, the present invention provides an oral and dental composition comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
   and a physiologically acceptable excipient.

The term “effective antibacterial amount” refers to an amount of one or more of the components in the composition sufficient to retard or prevent bacterial growth.

Preferably, chitosan or the chitosan derivative is in an encapsulated form such as in the form of microparticles or nanoparticles. A form of proprietory encapsulated chitosan such as supplied by Ellis & Associates, Victoria, Australia may be used.

Preferably, the chitosan or chitosan derivative is encapsulated in an aqueous solution at a pH of about 3.0 to 6.5. Preferably, the pH is about 3.0 to 4.0. Alternatively, the pH may be about 6.0 to 6.5.

Preferably, the encapsulated chitosan is present in the composition in an amount from about 0.5 to 2.0% by weight. Preferably, the encapsulated chitosan is present in an amount of 1% by weight.

Alternatively, chitosan or the chitosan derivative may be dissolved or dispersed in a physiologically acceptable aqueous solution such as a dilute aqueous acidic solution. The aqueous acidic solution may be acetic acid, ascorbic acid or citric acid. Preferably, the pH of the chitosan or chitosan derivative in the aqueous acid solution is about 3.0 to 4.0.

Preferably, the chitosan derivative is an N-chitosan derivative. The term “N-chitosan derivative” is to be understood to mean chitosan derivatives having alkyl or substituted alkyl groups bonded to the N-substituent of chitosan. Preferably such alkyl groups are C₁-C₆ and the N-chitosan derivative is present in the composition in an amount of 0.1 to 1 weight percent. Typically, in a non-encapsulated form, the N-chitosan derivative is present in an amount of 0.1 percent by weight, and in an encapsulated form the N-chitosan derivative is present in an amount of 1 percent by weight. The N-chitosan derivative may be N-carboxybutyl chitosan or N-carboxymethyl chitosan.

The term “physiologically acceptable divalent metal ion” refers to a divalent metal ion which is suitable for use in an oral or dental composition, that is which is not contraindicated for oral or dental use. Preferably, the divalent metal ion is Zn²⁺, Cu²⁺ or Sn²⁺, or the like.

The metal ion may be included in the composition in an amount from about 0.1% to 0.5% by weight, and more preferably in an amount from about 0.2% to 0.25% by weight. More preferably, the metal ion is present in the composition in an amount of about 0.22% by weight.

The divalent metal ion is preferably present in the composition in the form of a salt such as a chloride, acetate or sulphate. Preferably, the metal salt is water soluble and may be zinc chloride, zinc acetate, zinc sulphate or the like.

The term “quaternary ammonium compound” refers to any suitable quaternary ammonium compound used in oral and dental hygiene compositions. These may be C₈-C₁₈ alkyl pyridinium chlorides and C₈-C₁₈ alkyl pyridinium bromides. Preferably, the quaternary ammonium compound is cetyl pyridinium chloride.

Preferably, the quaternary ammonium compound is present in the composition in a range of about 0.01% to 0.1% by weight and more preferably about 0.05% by weight.

The excipient may take any suitable form such as a gel, a gelling agent such as a non-ionic gelling or thickening agent, or chitosan or the chitosan derivative may form the excipient. The gelling agent may be a natural or synthetic gum such as hydroxyethyl cellulose, hydroxymethyl cellulose and the like. The gelling agent may be present in the composition in an amount of about 0.5% to 5% by weight. Preferably the gelling agent is present in an amount of about 2% to 4% by weight, and more preferably in an amount of about 2.5% to 3% by weight.

The composition may be in the form of a gel, a mouthwash, a toothpaste, a chewing gum, a spray, incorporated in dental floss, or may form part of food.

The composition may comprise other suitable components to provide an appropriate oral or dental composition. These other components may comprise one or more non-ionic or cationic surfactants to provide a foaming action. Preferably, the surfactant is present in an amount of 0.1 to 5% by weight. Preferably the surfactant is a non-ionic surfactant or a betaine surfactant such as cocoamidopropylbetaine, lauramidopropylbetaine or cocobetaine.

Furthermore, fluoride or fluoride-providing compounds may also be added to the composition. Typically, a fluoride-providing compound may be present in an amount of about 0.005 to 2.5% by weight. The fluoride-providing compound may be a soluble inorganic fluoride salt such as sodium or potassium fluoride or sodium monofluorophosphate.

Other optional components may include anti-staining compounds (such as hydrogen peroxide), sweeteners (such as xylotol or sodium saccharine), colouring agents, flavouring agents and/or bleaching agents.

In a particularly preferred aspect, the invention provides an oral and dental composition comprising an effective antibacterial amount of a combination of:
a) a physiologically acceptable divalent ion selected from the group consisting of Zn²⁺, Cu²⁺ and Sn²⁺;
b) cetyl pryridinium chloride; and
c) chitosan or a chitosan derivative in a form to allow interaction with the other two components of the combination;
   and a physiologically acceptable excipient.

The composition of the invention may further comprise a thickening agent, a fluoride-providing compound, a non-ionic or a cationic surfactant, and an anti-staining agent.

The composition may also be incorporated in conventional toothpaste to provide antibacterial effects not usually provided in many toothpastes.

The invention has found particular application in the treatment of xerostomia (dry mouth). Xerostomia is a condition where the subject has a dry mouth due to insufficient saliva production. Xerostomia may occur after radiotherapy or chemotherapy, and is also associated with other conditions such as Sjorgren's syndrome.

Patients suffering from xerostomia have problems sleeping as it is often necessary for them to wet their mouths during the night. These patients also experience other oral and dental problems such as sensitive teeth, dry gums and mouth ulcers.

Thus, the present invention also provides an oral and dental composition for the treatment of xerostomia comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
   and a physiologically acceptable excipient.

The term “effective antibacterial amount” refers to an amount of one or more of the components in the composition sufficient to retard or prevent bacterial growth.

Preferably, chitosan or the chitosan derivative is in an encapsulated form such as in the form of microparticles or nanoparticles. A form of proprietory encapsulated chitosan such as supplied by Ellis & Associates, Victoria, Australia may be used.

Preferably, the chitosan or chitosan derivative is encapsulated in an aqueous solution at a pH of about 3.0 to 6.5. Preferably, the pH is about 3.0 to 4.0. Alternatively, the pH may be about 6.0 to 6.5.

Preferably, the encapsulated chitosan is present in the composition in an amount from about 0.5 to 2.0% by weight. Preferably, the encapsulated chitosan is present in an amount of 1% by weight.

Alternatively, chitosan or the chitosan derivative may be dissolved or dispersed in a physiologically acceptable aqueous solution such as a dilute aqueous acidic solution. The aqueous acidic solution may be acetic acid. Preferably, the pH of the chitosan or chitosan derivative in the aqueous acid solution is about 3.0 to 4.0.

Preferably, the chitosan derivative is an N-chitosan derivative. The term “N-chitosan derivative” is to be understood to mean chitosan derivatives having alkyl or substituted alkyl groups bonded to the N-substituent of chitosan such as N-carboxybutyl chitosan or N-carboxymethyl chitosan.

The term “physiologically acceptable divalent metal ion” refers to a divalent metal ion which is suitable for use in an oral or dental composition, that is which is not contraindicated for oral or dental use. Preferably, the divalent metal ion is Zn²⁺, Cu²⁺ or Sn²⁺, or the like.

The term “quaternary ammonium compound” refers to any suitable quaternary ammonium compound used in oral and dental hygiene compositions. These may be C8-C18 alkyl pyridinium chlorides and C8-C18 alkyl pyridinium bromides. Preferably, the quaternary ammonium compound is cetyl pyridinium chloride.

Preferably, the quaternary ammonium compound is present in the composition in a range of about 0.01% to 0.1% by weight and more preferably about 0.05% by weight.

The excipient may take any suitable form such as a gel, a gelling agent such as a non-ionic gelling or thickening agent, or chitosan or the chitosan derivative may form the excipient. The gelling agent may be a natural or synthetic gum such as hydroxyethyl cellulose, hydroxymethyl cellulose and the like. The gelling agent may be present in an amount of about 0.5% to 5% by weight in the composition.

Preferably the gelling agent is present in an amount of about 2% to 4% by weight, and more preferably in an amount of about 2.5% to 3% by weight.

The composition may comprise other suitable components to provide an appropriate oral or dental composition such as surfactants, flavouring and colouring agents.

The present invention further provides a method of inhibiting or preventing an oral or dental condition in a subject, the method comprising contacting an oral cavity and/or teeth of the subject with a composition comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
   and a physiologically acceptable excipient, for a time and under conditions sufficient to inhibit or prevent the condition.

The term “oral or dental condition” refers to one or more undesirable conditions of the mouth, tongue, teeth and/or gums such as mouth ulcers, Candida infections, dental plaque, calculus, dental caries (crown and root), gingivitis, xerostomia, tooth hypersensitivity, and the like.

The subject is to be understood to include not only humans but also animals such as dogs, cats, horses, cows, and the like. In the case of animal use, the composition may be provided in the form of a chew, a spray or a chewable novelty toy.

The time and conditions sufficient to inhibit or prevent the oral or dental condition will vary according to the extent of the oral or dental condition. Preferably, the composition is applied for a few minutes followed by rinsing of the oral cavity. Typically a time period of one to three minutes is suitable.

Alternatively, the composition may be allowed to remain in the mouth without rinsing it out to maximise the antibacterial effect. When the composition is provided in the form of a food, an outer coating, or contained within chewing gum or a toy (animal use), then the period of time taken to chew the food is usually sufficient.

In a particularly preferred aspect, the invention relates to a method of inhibiting or preventing xerostomia in a human or animal subject.

Accordingly, the present invention also provides a method of inhibiting or preventing xerostomia in a subject, the method comprising contacting an oral cavity and/or teeth of the subject with a composition comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
   and a physiologically acceptable excipient, for a time and under conditions sufficient to inhibit or prevent the condition.

Furthermore, the present invention provides an antibacterial oro-dental system comprising a composition including a physiologically acceptable divalent metal ion, a quaternary ammonium compound, chitosan or a chitosan derivative, a physiologically acceptable excipient, and saliva in the oral cavity of a human or animal subject, the chitosan or chitosan derivative present in a form to facilitate interaction with the components of the system including the saliva of the subject.

### BEST MODE(S) FOR CARRYING OUT THE INVENTION

An example of the invention will be described but it should be appreciated that the invention is not limited thereto and can be carried out in other forms.

An oral and dental composition according to the example comprises the following ingredients per kilogram:

0.5 g cetyl pyridinium chloride (CPC) BP

2.2 g zinc chloride BP

2.2 g sodium fluoride BP

2.5 g hydroxyethylcellulose (Natrosol 250H)

10 g encapsulated chitosan liquid (Ellis & Associates—pH 3.0 to 4.0; specific gravity of 0.970-1.3 gms/cc)

1.5 g methylhydroxybenzoate BP

4 g saccharin sodium BP

10 g Plantaren 1200UP surfactant

2.5 g spearmint flavour (BBA09-3103, Bush Boake Allen)

2.5 g peppermint flavour (BBA07-7686, Bush Boake Allen)

3 ml Orion green colour (code GRE00305S, 30 g/l)

3 ml Orion blue colour (code BLU0063S, 30 g/l)

Purified water to 1000 g

The composition is prepared as follows:

The purified water is added to a suitable mixing vessel. The CPC, zinc chloride, sodium fluoride, saccharin sodium, plantaren, flavouring and colouring are added sequentially to the water whilst stirring. The methylhydroxybenzoate is dissolved in additional flavouring and then added to the main batch. The hydroxyethyl cellulose is then added whilst stirring continuously. Finally, the encapsulated chitosan is added and stirring continued until all ingredients are fully dispersed.

Modifications and improvements may be made to the foregoing example without departing from the scope of the present invention. Furthermore, it should be appreciated that the scope of the invention is not limited to the scope of the example described.

Throughout the specification, unless the context requires otherwise, the word “comprise” or variations such as “comprises” or “comprising”, will be understood to imply the inclusion of a stated integer or group of integers but not the exclusion of any other integer or group of integers.

1. An oral and dental composition comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
and a physiologically acceptable excipient.

2. An oral and dental composition according to claim 1 wherein chitosan or the chitosan derivative is in an encapsulated form.

3. An oral and dental composition according to claim 2 wherein the chitosan or the chitosan derivative is in the form of microparticles.

4. An oral and dental composition according to claim 3 wherein the chitosan or the chitosan derivative is in the form of nanoparticles.

5. An oral and dental composition according to claim 2 wherein the chitosan or the chitosan derivative is encapsulated in an aqueous solution at a pH of about 3.0 to 6.5.

6. An oral and dental composition according to claim 5 wherein the pH is about 3.0 to 4.0.

7. An oral and dental composition according to claim 5 wherein the pH is about 6.0 to 6.5.

8. An oral and dental composition according to claim 2 wherein the chitosan or the chitosan derivative is present in the composition in an amount of about 0.5 to 2.0% by weight.

9. An oral and dental composition according to claim 8 wherein the chitosan or the chitosan derivative is present in the composition in an amount of about 1.0% by weight.

10. An oral and dental composition according to claim 1 wherein chitosan or the chitosan derivative is dissolved or dispersed in a physiologically acceptable aqueous solution.

11. An oral and dental composition according to claim 10 wherein the aqueous solution is a dilute aqueous acidic solution.

12. An oral and dental composition according to claim 11 wherein the dilute aqueous acidic solution is an acetic acid, ascorbic acid or citric acid solution.

13. An oral and dental composition according to claim 10 wherein the aqueous solution has a pH of about 3.0 to 4.0.

14. An oral and dental composition according to claim 1 wherein the chitosan derivative is an N-chitosan derivative.

15. An oral and dental composition according to claim 14 wherein the N-chitosan derivative comprises C1 to C6 alkyl or substituted alkyl groups attached to the N-substituent of the chitosan such as N-carboxybutyl chitosan or N-carboxymethyl chitosan.

16. An oral and dental composition according to claim 14 wherein the N-chitosan derivative is present in the composition in an amount of 0.1 to 1% by weight.

17. An oral and dental composition according to claim 16 wherein the N-chitosan derivative is present in an amount of 0.1% by weight when the N-chitosan is in a non-encapsulated form.

18. An oral and dental composition according to claim 16 wherein the N-chitosan derivative is present in an amount of 1.0% by weight when the N-chitosan is in an encapsulated form.

19. An oral and dental composition according to claim 1 wherein the divalent metal ion is chosen from the group consisting of Zn²⁺, Cu²⁺ and Sn²⁺.

20. An oral and dental composition according to claim 1 wherein the divalent metal ion is present in an amount from about 0.2% to 0.25% by weight.

21. An oral and dental composition according to claim 20 wherein the divalent metal ion is present in an amount of about 0.22% by weight.

22. An oral and dental composition according to claim 1 wherein the divalent metal ion is present in the form of a salt such as chloride, acetate or sulphate.

23. An oral and dental composition according to claim 22 wherein the divalent metal ion is present in the form of a salt such as zinc chloride, zinc acetate or zinc sulphate.

24. An oral and dental composition according to claim 1 wherein the quaternary ammonium compound is a (C8-C18) alkyl pyridinium chloride or a (C8-C18) alkyl pyridinium bromide.

25. An oral and dental composition according to claim 24 wherein the quaternary ammonium compound is cetyl pyridinium chloride.

26. An oral and dental composition according to claim 1 wherein the quaternary ammonium compound is present in the composition in an amount of 0.01% to 0.1% by weight.

27. An oral and dental composition according to claim 26 wherein the quaternary ammonium compound is present in the composition in an amount of 0.05% by weight.

28. An oral and dental composition according to claim 1 wherein the excipient is a gel, a gelling agent or a thickening agent.

29. An oral and dental composition according to claim 28 wherein the gelling agent is a non-ionic gelling or thickening agent.

30. An oral and dental composition according to claim 28 wherein the gelling agent is a natural or synthetic gum such as hydroxyethyl cellulose or hydroxymethyl cellulose.

31. An oral and dental composition according to claim 28 wherein the gelling agent is present in the composition in an amount of about 0.5% to 5% by weight.

32. An oral and dental composition according to claim 31 wherein the gelling agent is present in the composition in an amount of 2% to 4% by weight.

33. An oral and dental composition according to claim 31 wherein the gelling agent is present in the composition in an amount of 2.5% to 3% by weight.

34. An oral and dental composition according to claim 1 wherein the composition further comprises a non-ionic or cationic surfactant.

35. An oral and dental composition according to claim 34 wherein the surfactant is present in the composition in an amount of 0.1% to 5% by weight.

36. An oral and dental composition according to claim 34 wherein the surfactant is a surfactant such as cocoamidopropylbetaine, lauramidopropylbetaine or cocobetaine.

37. An oral and dental composition according to claim 1 wherein the composition further comprises fluoride or fluoride-providing compounds.

38. An oral and dental composition according to claim 37 wherein the fluoride-providing compound is present in the composition in an amount of about 0.005 to 2.5% by weight.

39. An oral and dental composition according to claim 37 wherein the fluoride-providing compound is a soluble inorganic fluoride salt such as sodium or potassium fluoride or sodium monofluorophosphate.

40. An oral and dental composition according to claim 1 wherein the composition further comprises anti-staining compounds, sweeteners, colouring agents, flavouring agents and/or bleaching agents.

41. An oral and dental composition comprising an effective antibacterial amount of a combination of:
a) a physiologically acceptable divalent ion selected from the group consisting of Zn²⁺, Cu²⁺ and Sn²⁺;
b) cetyl pryridinium chloride; and
c) chitosan or a chitosan derivative in a form to facilitate interaction with the other two components of the combination;
and a physiologically acceptable excipient.

42. An oral and dental composition for the treatment of xerostomia comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
and a physiologically acceptable excipient.

43. An oral and dental composition according to claim 42 wherein chitosan or the chitosan derivative is in an encapsulated form.

44. An oral and dental composition according to claim 43 wherein the chitosan or the chitosan derivative is in the form of microparticles.

45. An oral and dental composition according to claim 44 wherein the chitosan or the chitosan derivative is in the form of nanoparticles.

46. An oral and dental composition according to claim 43 wherein the chitosan or the chitosan derivative is encapsulated in an aqueous solution at a pH of about 3.0 to 6.5.

47. An oral and dental composition according to claim 46 wherein the pH is about 3.0 to 4.0.

48. An oral and dental composition according to claim 46 wherein the pH is about 6.0 to 6.5.

49. An oral and dental composition according to claim 43 wherein the chitosan or the chitosan derivative is present in the composition in an amount of about 0.5 to 2.0% by weight.

50. An oral and dental composition according to claim 49 wherein the chitosan or the chitosan derivative is present in the composition in an amount of about 1.0% by weight.

51. An oral and dental composition according to claim 42 wherein chitosan or the chitosan derivative is dissolved or dispersed in a physiologically acceptable aqueous solution.

52. An oral and dental composition according to claim 51 wherein the aqueous solution is a dilute aqueous acidic solution.

53. An oral and dental composition according to claim 52 wherein the dilute aqueous acidic solution is an acetic acid solution.

54. An oral and dental composition according to claim 52 wherein the aqueous solution has a pH of about 3.0 to 4.0.

55. An oral and dental composition according to claim 42 wherein the chitosan derivative is an N-chitosan derivative.

56. An oral and dental composition according to claim 55 wherein the N-chitosan derivative comprises C1 to C6 alkyl or substituted alkyl groups attached to the N-substituent of the chitosan such as N-carboxybutyl chitosan or N-carboxymethyl chitosan.

57. An oral and dental composition according to claim 55 wherein the N-chitosan derivative is present in the composition in an amount of 0.1 to 1% by weight.

58. An oral and dental composition according to claim 57 wherein the N-chitosan derivative is present in an amount of 0.1% by weight when the N-chitosan is in a non-encapsulated form.

59. An oral and dental composition according to claim 57 wherein the N-chitosan derivative is present in an amount of 1.0% by weight when the N-chitosan is in an encapsulated form.

60. An oral and dental composition according to claim 42 wherein the divalent metal ion is chosen from the group consisting of Zn²⁺, Cu²⁺ or Sn²⁺.

61. An oral and dental composition according to claim 42 wherein the divalent metal ion is present in an amount from about 0.2% to 0.25% by weight.

62. An oral and dental composition according to claim 61 wherein the divalent metal ion is present in an amount from about 0.22% by weight.

63. An oral and dental composition according to claim 42 wherein the divalent metal ion is present in the form of a salt such as chloride, acetate or sulphate.

64. An oral and dental composition according to claim 63 wherein the divalent metal ion is present in the form of a salt such as zinc chloride, zinc acetate or zinc sulphate.

65. An oral and dental composition according to claim 42 wherein the quaternary ammonium compound is a (C8-C18) alkyl pyridinium chloride or a (C8-C18) alkyl pyridinium bromide.

66. An oral and dental composition according to claim 65 wherein the quaternary ammonium compound is cetyl pyridinium chloride.

67. An oral and dental composition according to claim 42 wherein the quaternary ammonium compound is present in the composition in an amount of 0.01% to 0.1% by weight.

68. An oral and dental composition according to claim 67 wherein the quaternary ammonium compound is present in the composition in an amount of 0.05% by weight.

69. A method of inhibiting or preventing an oral or dental condition in a subject, the method comprising contacting an oral cavity and/or teeth of the subject with a composition comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
and a physiologically acceptable excipient, for a time and under conditions sufficient to inhibit or prevent the condition.

70. A method according to claim 69 wherein the composition is contacted with the oral cavity and/or teeth for one to three minutes.

71. A method of inhibiting or preventing xerostomia in a subject, the method comprising contacting an oral cavity and/or teeth of the subject with a composition comprising an effective antibacterial amount of a combination of:
a) a physiologically acceptable divalent metal ion;
b) a quaternary ammonium compound; and
c) chitosan or a chitosan derivative in a form to facilitate interaction with the other two components of the combination;
and a physiologically acceptable excipient, for a time and under conditions sufficient to inhibit or prevent the condition.

72. A method according to claim 71 wherein the composition is contacted with the oral cavity and/or teeth for one to three minutes.

73. An antibacterial oro-dental system comprising a composition including a physiologically acceptable divalent metal ion, a quaternary ammonium compound, chitosan or a chitosan derivative, a physiologically acceptable excipient, and saliva in the oral cavity of a human or animal subject, the chitosan or chitosan derivative present in a form to facilitate interaction with the components of the system including the saliva of the subject.
